(19) United States
(12) Patent Application Publication
(10) Pub. No.: US 2023/0104903 A1
(43) Pub. Date: Apr. 6, 2023

(54) Comprehensive approach to blood transfusion information analysis to ensure patient safety, optimal donor selection, red blood cell unit distribution optimization, and blood bank long-term inventory operational efficiency.

(71) Applicants: Gennady Peysakhovich, FEASTERVILLE TREVOSE, PA (US); Elena Butina, Kirov (RU); Garrett Jay Friedman, Kinnelon, NJ (US)

(72) Inventors: Gennady Peysakhovich, FEASTERVILLE TREVOSE, PA (US); Elena Butina, Kirov (RU); Garrett Jay Friedman, Kinnelon, NJ (US)

(21) Appl. No.: 17/449,802

(22) Filed: Oct. 1, 2021

(51) Int. Cl. G16H 20/00; G16H 10/60

(52) U.S. Cl. CPC G16H 20/00; G16H 10/60

(57) ABSTRACT

A comprehensive solution which introduces a blood transfusion recipient classification scheme and a method for recommending specific blood product units for transfusion candidates including those of complex cases, rare blood types and emergent situations for the achievement of: compatibility, safety and efficacy; prevention of alloimmunization and Hemolytic Transfusion Reactions; optimal patient to donor matching; and optimization of available red blood cell units distribution amongst recipients. This invention includes a method to quantify match results and measure outcomes of the decision-making processes. A donor phenotype classification scheme and a method to optimize the long-term storage of blood units are introduced.

### BACKGROUND OF THE INVENTION

### Field of the Invention

The invention relates to the field of Immunohematology, specifically to a blood transfusion and blood collection.

### Background

The most common procedure performed during a hospitalization in the United States is blood transfusion. In the US approximately 15 million red blood cell (RBC) units are transfused annually. Worldwide this number is approximately 85 million.^{1, 2}

A large amount of information is accumulated daily in electronic medical records (EMRs), laboratory information systems, and patient/donor databases. Both medical researchers and physicians are challenged with consuming so much information with size and complexity that exceeds the intellectual capacity of a person.

For each patient, a physician determines the depth of the immunohematological examination and selects blood products using clinical guidelines and standard operating procedures. In complex medical cases, however, there is a need to consider multi-factorial immunohematological and clinical parameters and this significantly complicates the medical decision-making process. In these cases, physicians must make such determinations relying on their own knowledge and experience.

This current selection practice poses significant challenges for the effective prevention of patient alloimmunization. Because working with a large amount of data and the existing standard methods to select red blood cells that have the desired repertoire of antigens requires a significant amount of time and resources. This is even more the case given the increased demand for ‘extended phenotype matching’. Furthermore, finding suitable blood products for recipients who either lack common antigens, or possess uncommon antigens/antigenic combinations, or have antibodies to common antigens is especially challenging.

Cryopreserved red blood cells are necessary when fresh red blood cells with the required phenotypes are not available in the blood bank and there is a lack of time to invite compatible donors. This situation occurs when a recipient has a rare blood type where in the phenotype there are no common antigens/antigenic combinations. Or where the recipient has antibodies to antigens commonly found in the general population. Or where an “extended phenotype matching” selection is required for patients who possess alloantibodies or autoantibodies.

Managing this reserve of cryopreserved red blood cells is a difficult task. Refrigerated red blood cells with phenotypes compatible with a large number of recipients are in high demand. Also, there are extremely rare phenotypes which are necessary only for “complex” recipients that may be rejected due to lack of demand. Filling the long-term inventory with (cryopreserved) red blood cells empirically, typically leads to the accumulation of red blood cells with phenotypes that are widespread in the population. This mirrors what is already available in a refrigerated form.

The art of managing blood supplies is maintaining a balance between the collection and distribution of blood components. Distributing red blood cells amongst patients in such a way so that each receives the required level of compatibility while minimizing wasted product requires complicated unit selection decisions. The more antigens of donors and recipients' phenotypes that need to be compared the more difficult these decisions are.

In a situation of not having blood units with the required level of compatibility in the blood bank inventory nor the time to wait for their acquisition, the physician is forced to select from partially compatible red blood cells. They currently do this by manually assessing the level of immunogenicity and clinical significance of various antigens. This may lead to alloimmunization which can develop into a serious problem requiring additional transfusion care resources and increasing the probability of future Hemolytic Transfusion Reactions (HTR).

Life-saving blood transfusions may carry some risk. Development of alloantibodies might occur in (RBC) transfusion recipients. This transfusion induced alloantibody formation is a clinically significant problem.

Alloimmunization is the process of antibody formation in response to an antigenic stimulation. It occurs when a foreign antigen that is not in the phenotype of a person enters their body. A foreign antigen is transmitted during a blood transfusion (or pregnancy) when the donor and recipient (or mother and fetus) are incompatible. The immune system of the recipient (or mother) produces alloantibodies which are specific to the antigen.

Blood transfusion recipient alloimmunization is a common adverse side effect of component therapy. The consequences of alloimmunization are acute or delayed Hemolytic Transfusion Reactions (HTR) and Hemolytic Disease of the Fetus and Newborn (HDFN).

According to the ‘Annual SHOT Report 2018 Summary’, hemolytic transfusion reactions caused by ABO, and non-ABO-antibodies are the 3rd most leading cause of fatal transfusion reactions.³

Without matching RBC antigens of blood donors to transfusion recipients that were diagnosed with hemoglobinopathy the reported prevalence of alloantibodies is 19-43% and 11-15% for those diagnosed with myelodysplastic syndrome (MDS)^{4,5,6,7,8,9, 10}

The effects of antibody interactions with antigens ranges from mild symptomatic anemia to life-threatening hemolytic reactions. In addition, the presence of alloantibodies impedes the timely provision of compatible RBC components to recipients.¹¹

Interestingly, according to an article published in Transfusion Medicine, Apr. 29, 2019 Transfusion-Related Red Blood Cell Alloantibodies: Induction and Consequences' the morbidity and mortality burden of RBC alloimmunization is likely underestimated.¹²

Compatibility of donor and recipient phenotypes may be limited to ABO, RhD, C/c, E/e and K (limited phenotype matching) or extended with the addition of Fya/Fyb, Jka/Jkb, S/s as well as other antigens (extended phenotype matching).^{13, 14} The practice of accurately comparing phenotypes makes routine blood transfusions more challenging for hospitals and blood services. Phenotype matching is generally applied only to certain patient groups as most recipients are transfused with only ABO and RhD compatible red blood cells.

In the United States and the United Kingdom, red blood cell selection for patients with hem oglobinopathy are performed using the antigens C/c, E/e and K in addition to ABO and RhD.^{10, 15} In the Netherlands, for patients with autoimmune hemolytic diseases, MDS, and clinically significant antibodies red blood cell selection is performed also using C/c, E/e and K in addition to ABO and RhD. For patients with hem oglobinopathy RBC compatible Fya and where possible, antigens Jkb and S/s are also considered.¹⁶

Operating models that currently support patient-blood unit selection have significant limitations. Also, current operating procedures do not support efficient long-term blood bank inventory management. These challenges are due to insufficient information standardization and stratification. A universal operating model (comprehensive approach) that can address these challenges does not exist.

### BRIEF SUMMARY OF THE INVENTION

The invention describes a method for recommending specific blood product units for transfusion candidates including those with rare blood types, complex clinical presentations, and emergent situations. One of the main objectives of the invention is the prevention of transfusion complications and alloimmunization.

To achieve those objectives the invention introduces a method to quantitatively define the match results of patient and donor compatibility and for the calculation of a patient blood unit selection score. This method was created to ensure the optimal level of compatibility between blood transfusion recipients and donors while optimizing blood bank product distribution.

Additionally, the invention introduces a method to quantify the outcome of a blood unit selection by comparing it to solution model recommendations. This in turn enables a measured comparison of unit selection decisions.

Furthermore, the invention introduces a method to optimize the long-term storage of a blood bank inventory to ensure that immunohematologically compatible blood components are available for recipients when fresh red blood cells with the required phenotypes are absent and there is a lack of time to obtain patient compatible units.

The invention framework introduces two (2) foundational classification schemes: Transfusion Risk Group (TRG), and Phenotype Usage Type (PUT) and a principal method: Match Score (MS).

Transfusion Risk Group (TRG) classifies patient and precisely defines required patient to donor matching conditions. TRG also provides a method to create and analyze the patient profile of a service area (collective patient profile).

Match Score (MS) is a method used to measure and compare patient-donor-unit selection decisions against system recommendations. MS also provides a capability to evaluate aggregated and timeline-based framework data.

Phenotype Usage Type (PUT) classifies donor phenotype. PUT classification facilitates blood bank long-term storage inventory management.

The invention summarizes the patient blood transfusion related story through the ‘Transfusion Risk Group’ (TRG) classification scheme. This classification explains what information should be considered when evaluating transfusion safety and efficacy. TRG combines relevant patient demographics and transfusion conditions. TRG classifies patients into groupings, indicating the required donor selection level by phenotype.

When a red blood cell request is received the invention assigns a Transfusion Risk Group (TRG) by analyzing demographic, clinical, and laboratory information. Using this assigned TRG the invention then determines the level and specifics of recipient blood typing required for donor selection to ensure a safe transfusion and the prevention of the undesirable effects of alloimmunization.

A donor-recipient pair is considered compatible when a donor phenotype has no antigens which are not present in the recipient phenotype as per recipient's TRG. Compatibility includes the notion of an “identical antigen set” (donor and recipient have the same set of antigens). When selecting red blood cells for recipients, preference is given to donors with an “identical antigen set”. This approach avoids post-transfusion chimerism and keeps units that have fewer phenotype antigens in the blood bank so that they may be available for other patients.

The invention determines “Patient-Donor Compatibility” by matching patient to donor phenotypes based on the required information as specified by the recipient's Transfusion Risk Group (TRG). The solution ranks antigens in accordance with their immunogenicity (ability to activate antibody production in the recipient's body) and clinical significance (ability to cause transfusion reactions and hemolytic disease of the fetus and newborn).

Match Score (MS) is a method to compare multiple donors to a patient and to determine the best match. It can also be used in critical situations to determine “the best” incompatible donor. The MS method is used to evaluate patient-donor compatibility. Patient donor match result is represented in Match Score definitions: 1) the degree of matching (exact match, sufficient match, mismatch), 2) the numeric measurement of the match result, 3) deviation (number of rules) from an exact match, and 4) the number of antigen rules to apply in the matching algorithm.

When evaluating a patient to RBC blood unit match (unit) specific factors such as storage method, and expiration date should be taken into consideration in addition to the patient donor match result. Each unit specific factor is estimated using the MS method. The patient to blood unit match result is measured by adding unit additional factors to the patient donor match result. The invention compares multiple units to a patient and determines the best match. This process optimizes the decision-making task for multiple patients by assigning a recommended unit priority ranking to each patient unit pair.

The invention supports RBC unit selection using two models: 1) Recommended Selection: uses the required medical information as defined by the patient's TRG and additional RBC unit factors, 2) Analyzed Selection: uses all patient available medical information and additional RBC unit factors.

Cryopreservation, storage and thawing of blood components are expensive procedures. Therefore, red blood cells from a long-term storage bank are transfused only in special cases where there is an absence of compatible refrigerated red blood cells and timing does not permit inviting suitable donors. Cryopreservation blood bank also provides a repository of red blood cells whose phenotype does not contain antigens or combinations of antigens often found in the population.

The blood bank long-term component storage challenge is to determine the optimal size and antigen composition of the cryo-preserved blood units pool, which maximizes the probability of finding required red blood cells and minimizes unused RBC unit storage costs. Accordingly, the invention recognizes phenotypes and antigen combinations that are optimal for the selection of the largest number of recipients. In addition, the invention identifies RBC units that contain common or rare antigen combinations in donor phenotypes and recommends these units for long term storage. So that these blood components may be available to fulfill the critical needs of certain phenotype recipients.

When managing a long-term storage reserve, it is necessary to maintain an equilibrium between a high probability of finding red blood cell phenotypes and the excessive accumulation of red blood cells that go unclaimed and ultimately expire.

The Phenotype Usage Type (PUT) classification scheme is introduced to assist in solving this challenge. PUT describes how RBC blood units should be managed. The PUT classification introduces the Unique category and identifies the phenotypes that belong to it. There are eighty (80) unique phenotypes which are present for only a small number of donors. However, Unique phenotypes are compatible with 99.8% of recipients. The recommended standard of care of the Unique PUT category is to cryopreserve these RBC units. The complete PUT classification scheme includes Unique, Extraordinary, Required, Universal, and Common categories.

The invention's logical framework (a comprehensive approach) should prove to be beneficial to research laboratories that are involved in the field of immunohematology as well as to medical facilities, blood banks, physicians, medical device manufacturers and health quality assurance organizations.

### BRIEF DESCRIPTION OF DRAWINGS

FIG. 1 is a chart of Transfusion Risk Group Classification.

FIG. 2A is a flowchart diagram that describes the Profiles Creation process, part of the method for recommending specific blood product units for transfusion.

FIG. 2B is a flowchart diagram that describes the Patient Matching and Systems Recommendations process, part of the method for recommending specific blood product units for transfusion

FIG. 2C, is a flowchart diagram that describes the Patient Unit Selection process, part of the method for recommending specific blood product units for transfusion

FIG. 2D is a flowchart diagram that describes the Patient Unit Selection Review process, part of the method for recommending specific blood product units for transfusion

FIGS. 3A, 3B, 3C, and 3D are charts of Phenotype Usage Type of the classification Unique. These Figures show the combinations of antigens that determine the Unique PUT Category.

FIG. 4 is a chart of Phenotype Usage Type of the classification Required. Figure shows the combinations of antigens that determine the Required PUT Category.

FIG. 5 is a chart of Phenotype Usage Type of the classification Universal. Figure shows the combinations of antigens that determine the Universal PUT Category.

FIG. 6 is a chart of Phenotype Usage Type of the classification Extraordinary. Figure shows the combinations of antigens that determine the Extraordinary PUT Category.

FIG. 7 is a Claims Dependency Map. Figure shows how invention claims are related.

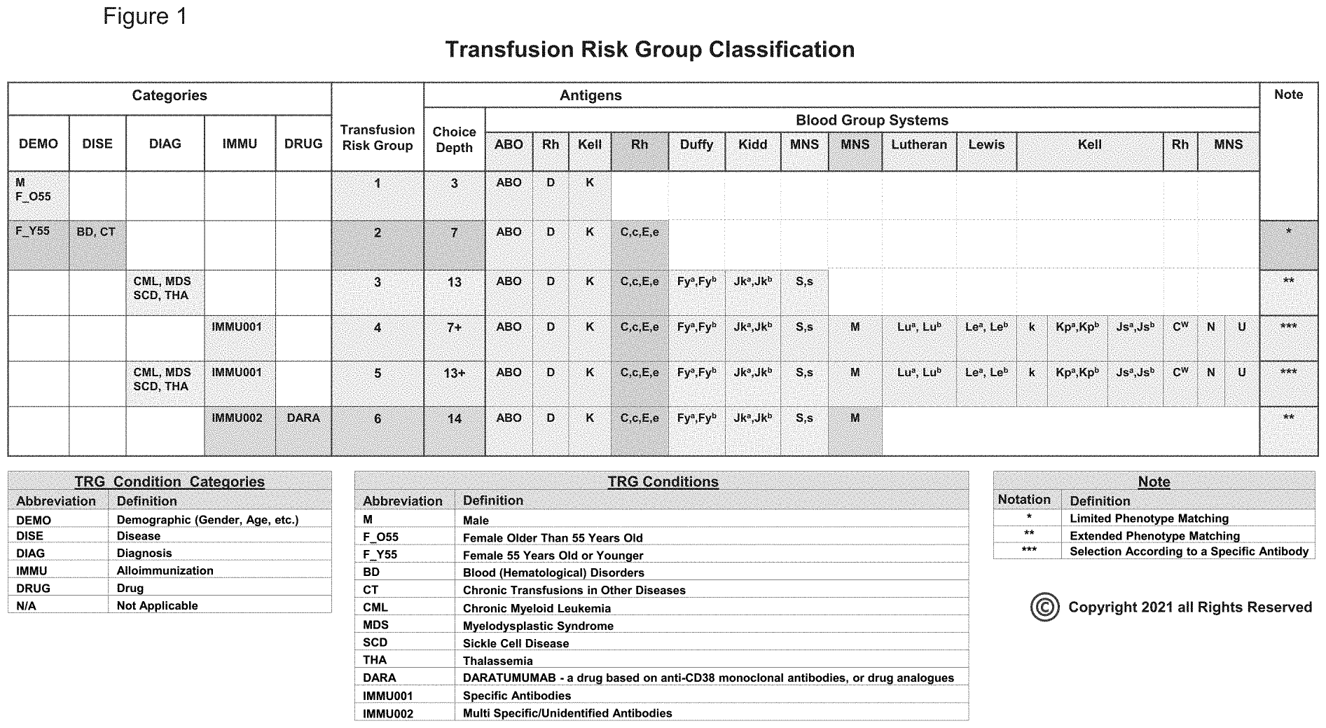

FIG. 1— Transfusion Risk Group (TRG) classifies patient and precisely defines required patient to donor matching conditions. Transfusion Risk Group (TRG) is a classification scheme that combines relevant patient demographics and transfusion conditions. Transfusion Risk Groups (TRG) 1,2,3,6 are defined according to the required antigen level matching rules. TRG Groups defined as 4 and 5 additionally consider unexpected RBC (specific) antibodies. These rules are used by the patient to donor matching process. Transfusion Risk Group (TRG) classifies patients into groups based on three categories a) relevant demographic factors: age and gender, b) related medical conditions and c) the presence of unexpected specific antibodies. TRG defines the antigens required for patient and donor matching. The TRG classifications are enumerated 0-6.

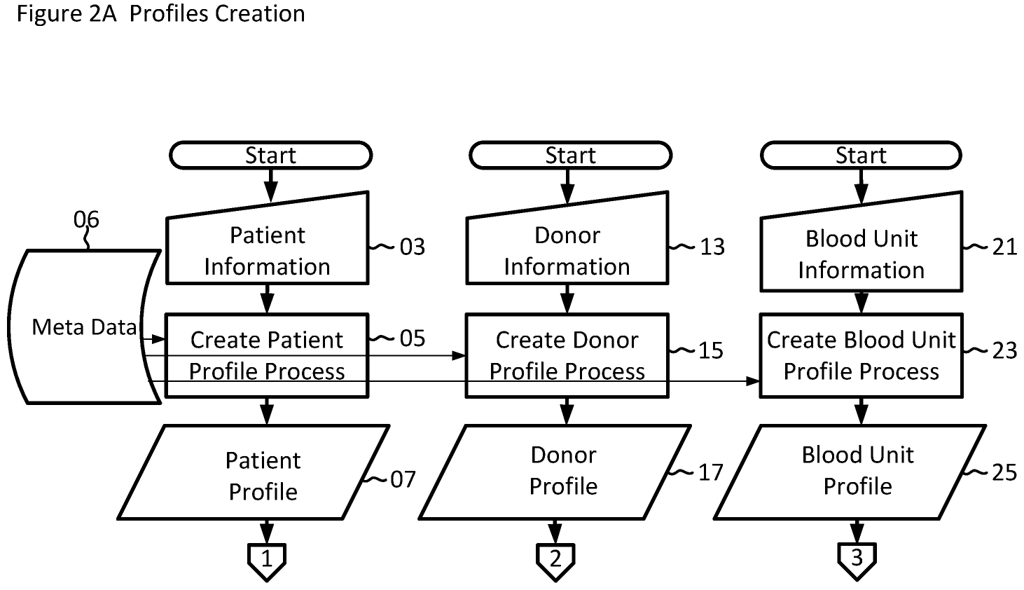

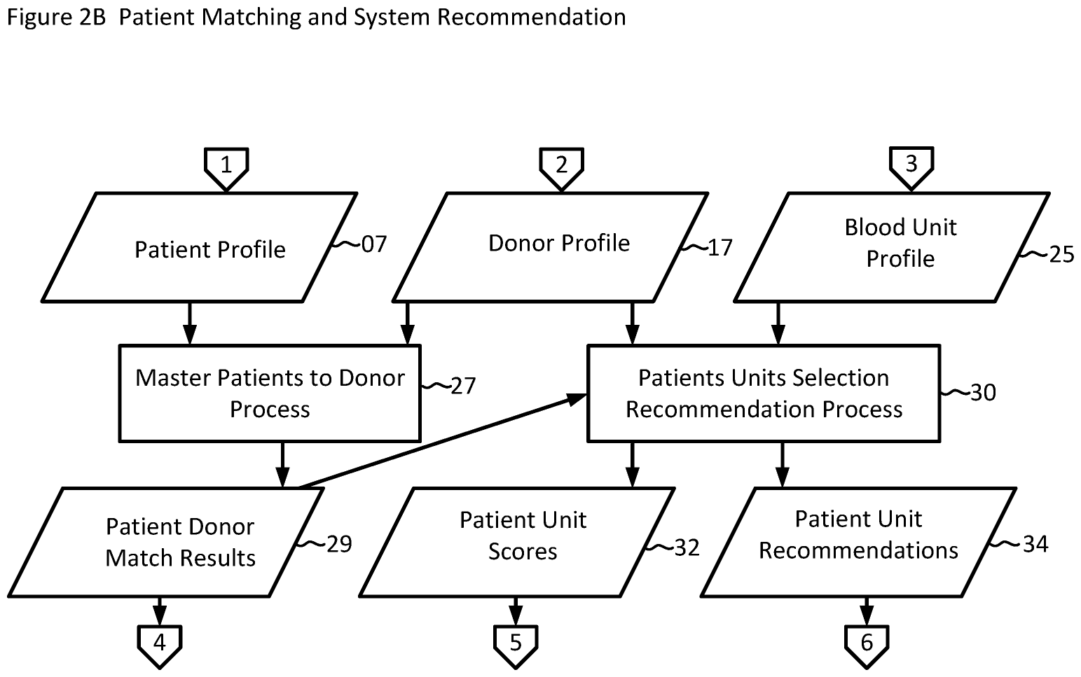

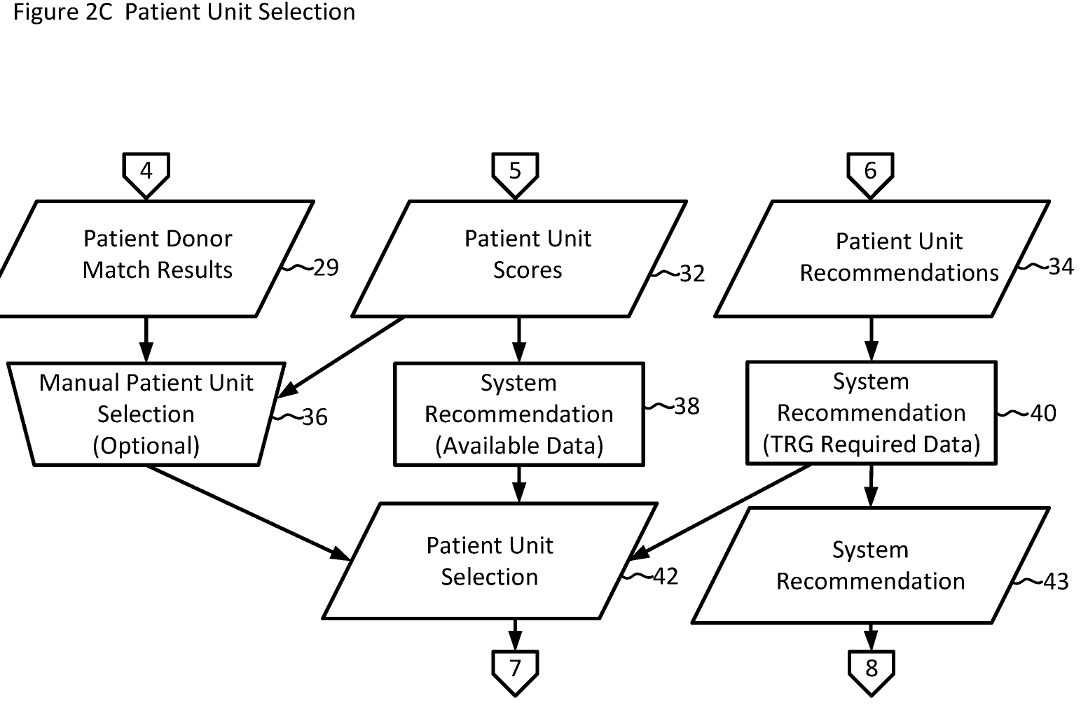

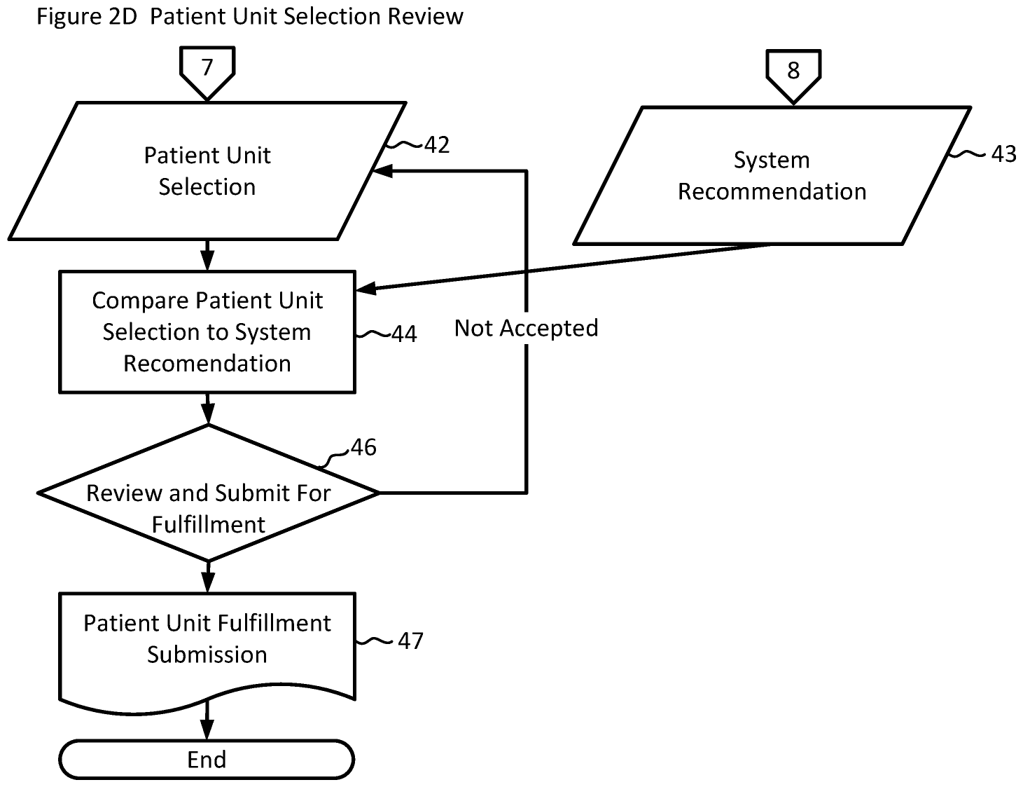

FIGS. 2A, 2B, 2C, and 2D— Flow chart illustrates a method for optimal red blood cell (RBC) unit patient recommendation from a blood bank's inventory or multiple blood bank inventories. The process flow of optimal unit recommendation is intended to be illustrative and may be implemented executing some or all the steps of the method with or without electronic processing devices.

Item 03—The invention receives patient information. It contains name (not required), date of birth (DOB) or age, gender, race, location (not required), facility patient id (not required), treatment facility (not required), transfusion conditions and relevant ICD10 codes, blood typing result (patient phenotype), and red blood cells unexpected antibodies (if applicable).

Step 05—Process creates ‘Patient Profile’ using available patient information and Transfusion Risk Group (TRG) classification. When a red blood cell request is received the invention analyzes demographic, clinical, and laboratory information about the recipient. In accordance with the Transfusion Risk Group (TRG) classification the following recipient parameters are analyzed: demographics (gender, age, race), clinical (transfusion conditions and relevant ICD10 codes), and laboratory (presence of unexpected specific and multispecific/pan agglutinating antibodies).

Item 06—Reference data that is used by Steps 05, 15, 23. Includes: a) Transfusion Risk Group (TRG) definitions, b) Phenotype Usage Type (PUT) classifications, c) Match Score (MS) Priority Factor values d) Information for assessing rare and impossible antigen combinations.

Item 07—Patient profile created in Step 05 of FIG. 2A contains: 1) Demographics, clinical and laboratory information, 2) Transfusion condition, derived for the relevant reported ICD10 code, 3) Assigned Transfusion Risk Group (TRG), 4) Required level and specifics of recipient blood typing for proper donor selection, 5) Blood typing information sufficiency, 6) Impossible, improbable, and rare antigen combinations and Chimerism condition.

Item 13—The invention receives donor information. It contains name (not required), date of birth (DOB) or age (not required), gender (not required), race, location (not required), and donor identification number (not required), donor's red blood cell units currently stored in a blood bank (if available), and blood typing result (donor phenotype).

Step 15—Process creates ‘Donor Profile’ using available donor information and Phenotype Usage Type (PUT) classification. Donor is assigned a Phenotype Usage Type (PUT) by matching a donor phenotype to the PUT definitions. Donor phenotype is checked for impossible, improbable, and rare antigen combinations.

Item 17—Donor profile created in step 15 of FIG. 2A contains: 1) Demographics, 2) Phenotype information, 3) Phenotype Usage Type (PUT) 4) Phenotype check result of impossible, improbable, or rare antigen combination 5) Invention recommended donor's blood unit ‘Standard of Care’ (storage method).

Item 21— The invention receives blood unit information. It contains unit identifiers, product description, storage location (blood bank), creation date and expiration date, current storage method, donor.

Step 23— Process creates blood unit profile. Unit inherits donor phenotype and phenotype usage type (PUT). The invention validates the current storage method against standard-of-care (storage method) recommendation and identifies discrepancy. Invention defines unit specific factors such as long-term storage method, Extraordinary PUT, and Expiration Date in MS Priority Factor values.

Item 25— Blood unit profile created in step 23 of FIG. 2A contains: 1) Blood bank identifier, Unit ID, Donor identifier, 2) Assigned phenotype usage type (PUT), 3) Current storage method, 4) Recommended storage method, 5) Storage method discrepancy indicator, 6) Unit start date, unit expiration date, 7) Unit specific factors such as long-term storage method, Extraordinary PUT, and Expiration Date all expressed in MS Priority Factor values.

Step 27— Utilizing Patient Profile and Donor Profile information predefined match rules are applied against all active donors. For each patient donor pair 1) Patient/Donor Compatibility is determined based on the required information as specified by recipient's Transfusion Risk Group (TRG) and 2) Patient/Donor Match is determined based on all available information. Match Score method is used to evaluate and measure patient donor pair results.

Item 29— Patient donor match result created in step 27 of FIG. 2B contains Patient Donor Compatibility and Patient Donor Match results represented using the inventions' Match Score (MS) method parameters: Match Score, Priority Factor, Choice Factor and Choice Depth.

Step 30— Utilizing donor profile information, labeled object 17 in FIG. 2B, blood unit profile information, labeled object 25 in FIG. 2B, and patient donor match result, labeled object 29 in FIG. 2B Patient Unit Selection Recommendation Process is performed. The patient to blood unit match result is measured by adding unit additional factors to the patient donor match result. The invention compares multiple blood units to a patient and determines the best match. This process optimizes the decision-making task for multiple patients by assigning a recommended unit priority ranking to each patient unit pair.

Item 32— Patient Unit Score is represented using the inventions' Match Score (MS) method parameters: Match Score, Priority Factor, Choice Factor and Choice Depth.

Item 34— The output of Patient Unit Selection Recommendation process step 30 in FIG. 2B. Patient Unit Recommendation is the best patient unit match (highest ranking unit for a patient). Units that are ‘In bank inventory’ but claimed by another patient are not included.

Step 36— Utilizing patient donor match results, labeled object 29 in FIG. 2C and patient unit scores, labeled object 32 in FIG. 2C Manual Patient Unit Selection Process is performed. The user applies patient to donor matching and unit specific factor filters to manually select a blood unit. An evaluation of the manual selection includes unit compatibility level, priority factor, rank, and other details. A comparison of the manually selected red blood cell unit is displayed against the invention's recommendation.

Step 38— Utilizing Patient Unit Scores, labeled object 32 in FIG. 2C and Patient Unit Recommendations, labeled object 34 in FIG. 2C Invention Recommendation (All Available Patient Data) Process is performed. The highest-ranking available red blood cell units according to Match Score Code and Priority Factor for a patient are presented for manual selection.

Step 40— Utilizing Patient Unit Recommendations, labeled object 34 in FIG. 2C Invention Recommendation (TRG Based Situationally Required Data) Process is performed. The highest-ranking available unit is recommended.

Step 42— Utilizing Manual Patient Unit Selection, labeled object 36 in FIG. 2C, Invention Recommendation, labeled object 38 in FIG. 2C, and Invention Recommendation, labeled object 40 in Figure Patient Unit Selection is performed. Unit is selected.

Item 43— Invention Recommendation created in Step 40 of FIG. 2C, Invention Recommendation (TRG Based Situationally Required Data) contains: 1) Patient Unit, 2) Patient unit recommendation match score parameters.

Step 44— Utilizing Patient Unit Selection labeled item 42 in FIG. 2D and Invention Recommendation labeled item 40 in FIG. 2D Compare Patient Unit Selection to Invention Recommendation Process is performed. Units are presented for user to verify selection.

Step 46— Utilizing Compare Patient Unit Selection to Invention Recommendation labeled item 44 in FIG. 2D Review and Submit Patient Unit for Fulfillment Process is performed. An option to submit the unit selection to a fulfillment invention, or to not accept and return to the patient unit selection step 42 is presented to the user.

Item 47— Accepted Patient Unit Submission.

### INVENTION DETAILED DESCRIPTION

The invention introduces three (3) foundational concepts—two (2) classification schemes: Transfusion Risk Group (TRG), and Phenotype Usage Type (PUT) and a method: Match Score (MS).

Section 1: The Transfusion Risk Group (TRG) Classification

One of the main functions of the invention is to prevent the emergence of clinically important antibodies (alloimmunization) resulting from blood transfusions. When selecting donors, the objective is not only to consider current transfusions but also those in the future. It is important that the donor selection be performed in a way that prevents the formation of unexpected antibodies so that the patient does not transition to an alloimmunized state. To achieve this, recipients are assigned to risk groups.

Transfusion Risk Group (TRG) is a classification scheme that combines relevant patient demographics and transfusion conditions.

Transfusion Risk Group (TRG) classifies patients into groups based on three categories a) demographic factors, b) clinical; related medical conditions, and c) laboratory; the presence of unexpected specific antibodies.

TRG defines the antigens required for patient and donor matching.

In accordance with the TRG classification the following recipient parameters are analyzed:

Demographic: gender, age, and race. Special attention to young women whose alloimmunization prevention is targeted to prevention of hemolytic disease of fetus and newborn.

Clinical: diagnosis, chronic transfusion of blood components, taking of drugs based on monoclonal antibodies to CD-38. Special attention is required for recipients with blood system diseases (primarily with diagnoses of MDS, CML, hemoglobinopathy) and/or long-term transfusion therapy which require the selection of red blood cells for the prevention of hemolytic transfusion reactions. Recipients taking drugs that cause nonspecific red blood cell agglutination require extended phenotype-matching donor selection.

Laboratory: presence of specific, nonspecific and multispecific (pan agglutinating) antibodies. If specific alloantibodies are detected in the recipient, selection is carried out using red blood cells in the phenotype where there is no antigen to which the patient's antibodies are directed. If nonspecific and/or pan agglutinating antibodies are detected, selection is carried out according to the “extended phenotype-matching” principle.

There are seven (7) Transfusion Risk Groups (TRGs) defined. Each TRG is associated with an algorithm to determine if a patient's blood typing (antigen) information is sufficient for donor matching and with an algorithm to perform patient donor matching.

The TRG classifications are enumerated 0-6. Transfusion Risk Groups (TRG) 1,2,3, and 6 are defined according to the required antigen level matching rules. TRG Groups 4, and 5 additionally consider unexpected RBC (specific) antibodies. These rules are used by the patient to donor matching process.

Transfusion Risk Group 1 (TRG 1)

Definition: The prevention of alloimmunization is limited only to transfusion-dangerous antigens: ABO, D and K. Donor selection that makes use of a greater number of antigens does not have clinical significance and increases the cost of the transfusion. These patients likely will not receive subsequent transfusions or have a change in their transfusion conditions.

Criteria: All Males and Females 55 years and older who do not possess any negative transfusion conditions

Phenotype: ABO, D, K (3 Antigen Rules)

Antibody: N/A (antibodies not found)

Transfusion Risk Group 2 (TRG 2)

Definition: Patients have a high probability of future transfusions or pregnancies. The prevention of alloimmunization in addition to ABO, D and K includes the C, c, E, e antigens.

Criteria: Females younger than 55, blood (Hematological) disorders, chronic transfusions due to other diseases.

Phenotype: ABO, D, K, C, c, E, e (7 Antigen Rules)

Antibody: N/A (antibodies not found)

Transfusion Risk Group 3 (TRG 3)

Definition: Lifelong blood transfusion dependency. It is a better course of action to prevent alloimmunization rather than to fight against the consequences it creates. Donor selection should be based on 13 antigens. Patient blood typing should be performed accordingly to this level.

Criteria: Disease diagnosis such as Sickle Cell Disease, Thalassemia, Chronic

Myeloid Leukemia, and Myelodysplastic Syndrome.

Phenotype: ABO, D, K, C, c, E, e, Fy^{a}, Fy^{b}, Jk^{a}, Jk^{b}, S, s (13 Antigen Rules)

Antibody: N/A (antibodies not found)

Transfusion Risk Group 4 (TRG 4)

Definition: The patient has unexpected specific RBC antibodies. Donor selection must consider both a base of seven (7) antigens (same as TRG 2) as well as additional antigens corresponding to the patient's antibodies.

Criteria: Presence of unexpected specific RBC antibodies.

Phenotype: ABO, D, K, C, c, E, e (7 Antigen Rules)+specific antibodies

Antibody: Specific antibodies including anti−D, −C, −c, −E, −e, −K, −k, −Fy^{a}, −Fy^{b}, −Jk^{a}, −Jk^{b}, −S, −s, −M, −N, −Lu^{a}, −Lu^{b}, −Le^{a}, −Le^{b}, −Kp^{a}, −Kp^{b}, −Js^{a}, −Js^{b}, −C^{w} and others.

Transfusion Risk Group 5 (TRG 5)

Definition: The patient has unexpected specific RBC antibodies and additional transfusion conditions. Donor selection must consider both a base of thirteen (13) antigens (same as TRG 3) as well as additional antigens that correspond to the patient's antibodies.

Criteria: Presence of unexpected specific RBC antibodies and disease diagnosis such as Sickle Cell Disease, Thalassemia, Chronic Myeloid Leukemia, and Myelodysplastic Syndrome

Phenotype: ABO, D, K, C, c, E, e, Fy^{a}, Fy^{b}, Jk^{a}, Jk^{b}, S, s (13 Antigen Rules)+specific antibodies

Antibody: Specific antibodies including anti−D, −C, −c, −E, −e, −K, −k, −Fy^{a}, −Fy^{b}, −Jk^{a}, −Jk^{b}, −S, −s, −M, −N, −Lu^{a}, −Lu^{b}, −Le^{a}, −Le^{b}, −Kp^{a}, −Kp^{b}, −Js^{a}, −Js^{b}, −C^{w} and others.

Transfusion Risk Group 6 (TRG 6)

Definition: Patients have pan-reactive antibodies, possibly introduced from prescribed medicines. These antibodies interact with the RBC of all donors. Donor selection should be based on 14 transfusion dangerous antigens.

Criteria: Presence of multispecific/unidentified antibodies

Phenotype: ABO, D, K, C, c, E, e, Fy^{a}, Fy^{b}, Jk^{a}, Jk^{b}, S, s, M (14 Antigen Rules) Antibody: Multispecific/unidentified antibodies

Transfusion Risk Group 0 (TRG 0)

Definition: Immediate transfusion, patient phenotype is unknown.

Donor phenotype must be O, D−C−c+E−e+, K− (O,ccdee,K−)

Criteria: Immediate transfusion, phenotype unknown or matching donor (unit) is not available for ABO, Rh

Phenotype: N/A (not applicable)

Antibody: N/A (not found or no information available)

Transfusion Risk Group (TRG) Examples

### EXAMPLE: 1

Transfusion Risk Group 1, Sufficient
information for donor selection

| | |
|---|---|
| Patient | ID = 6; Name: Demo Patient; |
| | Gender: Male; Age: 24 |
| Transfusion Conditions | Male, no negative transfusion |
| | conditions |
| Patient Phenotype | 3 antigens known |
| Assigned Transfusion Risk Group | TRG 1 |
| Messages | Sufficient information for donor |
| | selection |
| Choice Depth Recommendation: | 3 |
| TRG 1 required phenotype | ABO, D, K |
| | 3 antigen rules |

### EXAMPLE: 2

Transfusion Risk Group 2, Sufficient
information for donor selection

| | |
|---|---|
| Patient | ID = 2; Name: Demo Patient; |
| | Gender: Female; Age: 22 |
| Transfusion Conditions | Female, younger than 55 |
| Patient Phenotype | 9 antigens known |
| Assigned Transfusion Risk Group | TRG 2 |
| Messages | Sufficient information for donor |
| | selection |
| Choice Depth Recommendation: | 7 |
| TRG 2 required phenotype | ABO, D, K, C, c, E, e |
| | 7 antigen rules |

### EXAMPLE: 3

Transfusion Risk Group 3, Insufficient
information for donor selection

| | |
|---|---|
| Patient | ID = 10; Name: Demo Patient; |
| | Gender: Female; Age: 61 |
| Transfusion Conditions | CML—Chronic myeloid leukemia |
| Patient Phenotype | 15 antigens known |
| Assigned Transfusion Risk Group | TRG 3 |
| Messages: | Insufficient information for donor |
| | selection missing antigens Jk^{a} and Jk^{b} |
| Choice Depth Recommendation: | 13 |
| TRG 3 required phenotype | ABO, D, K, C, c, E, e, Fy^{a}, Fy^{b}, Jk^{a}, |
| | Jk^{b}, S, s |
| | 13 antigen rules |

### EXAMPLE: 4

Transfusion Risk Group 4, Sufficient
Information for donor selection

| | |
|---|---|
| Patient | ID = 13; Name: Demo Patient; |
| | Gender: Male; Age: 56 |
| Transfusion Conditions | Anti-Jk^{a} specific antibody |
| Patient Phenotype | 11 antigens known |
| Assigned Transfusion Risk Group | TRG 4 |
| Messages | Sufficient information for donor |
| | selection |
| Choice Depth Recommendation: | 7 + 1 = 8 |
| TRG 4 required phenotype | ABO, D, K, C, c, E, e + Anti-Jk^{a} |
| | 7 antigen rules + 1 specific antibody |

### EXAMPLE: 5

Transfusion Risk Group 5, Sufficient
information for donor selection

| | |
|---|---|
| Patient | ID = 14; Name: Demo Patient; |
| | Gender: Male; Age: 38 |
| Transfusion Conditions | CML—Chronic myeloid leukemia, |
| | Anti-Le^{a} specific antibody |
| Patient Phenotype | 17 antigens known |
| Assigned Transfusion Risk Group | TRG 5 |
| Messages | Sufficient information for donor |
| | selection |
| Choice Depth Recommendation: | 13 + 1 = 14 |
| TRG 5 required phenotype | ABO, D, K, C, c, E, e, Fy^{a}, Fy^{b}, Jk^{a}, |
| | Jk^{b}, S, s + Anti-Le^{a} |
| | 13 antigen rules + 1 specific antibody |

### EXAMPLE: 6

Transfusion Risk Group 6, Sufficient
information for donor selection

| | |
|---|---|
| Patient | ID = 18; Name: Demo Patient; |
| | Gender: Male; Age: 62 |
| Transfusion Conditions | BD—Blood (Hematological) |
| | disorders, DARA—Daratumumab |
| Patient Phenotype | 17 antigens known |
| Assigned Transfusion Risk Group | TRG 6 |
| System Messages | Sufficient information for donor |
| | selection |
| Choice Depth Recommendation: | 14 antigens |
| TRG 6 required phenotype | ABO, D, K, C, c, E, e, Fy^{a}, Fy^{b}, Jk^{a}, |
| | Jk^{b}, S, s, M |
| | 14 antigen rules |

Section 2: Patient Profile Creation

In accordance with the Transfusion Risk Group (TRG) classification the following recipient parameters are analyzed: demographics (gender, age, race), clinical (transfusion conditions and relevant ICD10 codes), and laboratory (presence of unexpected specific and multispecific/pan agglutinating antibodies).

Patient Profile contains the following:

1) Demographics, clinical and laboratory information.

2) Transfusion condition, derived for the relevant reported ICD10 code.

3) Assigned Transfusion Risk Group (TRG), determined by matching patient information to TRG classification.

4) Phenotype

5) Required level and specifics of recipient blood typing, for proper donor selection to ensure a safe transfusion and prevention of undesirable effects of alloimmunization.

6) Blood typing information sufficiency, evaluated against TRG requirements.

7) Impossible, improbable, and rare antigen combinations and Chimerism condition

Section 3: Patient Donor Matching

Patient Donor Compatibility is determined based on the required recipient information as specified by their Transfusion Risk Group (TRG). A donor-recipient pair is considered compatible when a donor phenotype has no antigens which are not present in the recipient phenotype as per recipient’ TRG.

A donor-recipient pair is considered incompatible when a donor phenotype has antigens which are not present in the recipient phenotype as per recipient's TRG. The Match Score method is used to evaluate and measure patient donor compatibility.

The patient donor match is determined based on all available information.

Compatibility includes the aspect of an “identical antigen set”. In this circumstance the donor and recipient have the same set of antigens. When selecting red blood cells for a recipient, preference should be given to an “identical antigen set” donor. This approach avoids post-transfusion chimerism and keeps units that possess less antigens in the phenotype in the blood bank as these units may be needed for other patients who have similar phenotypes.

The invention applies predefined match rules for all known relevant patient information against all active donors in the system.

Match rules are grouped into categories: Demographic, Phenotype, Antibody, and Compatibility.

Demographic, Phenotype and Antibody categories match rules are used to compare patient relevant information to available donor information at a fine grain level. The match rule's companion algorithms are applied for each rule accordingly. Algorithms use the MS Method to represent the result.

Compatibility category match rules are used to match patient to donor at the ‘person level’.

Patient Donor Matching conclusions are drawn independently for each antigen pair, required combination of antigens as per a recipient's TRG, and for the entire recipient phenotype. The invention's matching algorithm considers the presence of specific alloantibodies and autoantibodies, as well as the condition of Chimerism. Additionally, the invention evaluates demographic information (donor and recipient race), as well as patient gender and age when it is applicable.

Section 4: The Match Score (MS) Method

Match Score (MS) Method provides the capability to quantitatively define the match results of patient donor compatibility, calculate patient unit selection score, measure, and compare unit selection decisions to ensure the optimal level of compatibility between blood transfusion recipients and donors while optimizing blood products distribution.

The MS method is expressed by a Match Score Code and is clarified by the Priority Factor. Additionally, supplemental Choice Factor, and Choice Depth parameters are established in the method.

Match Score Code represents the degree of matching.

| | |
|---|---|
| Match Score Codes: | MS0 - Exact Match |
| | MS1 - Sufficient Match |
| | MSM - Mismatch |

Priority Factor is a numerical measurement of a) an executed fine grain match rule result, b) a patient donor compatibility, c) a patient donor match d) a patient unit selection score, e) blood unit specific factors, and f) patient demographic relevant characteristics.

A lower Priority Factor is given a higher priority in donor unit selection.

Choice Factor defines the deviation (number of rules) from an exact match.

Choice Depth defines the number of antigen rules to apply in a matching algorithm.

Section 5: Match Rules in Match Score (MS) Method Definitions

Patient donor matching phenotype category rule evaluation has one of the following four outcomes: a) Exact match—patient and donor antigen presence is identical (e.g., patient E−, donor E−; patient E+, donor E+), b) Sufficient match—patient and donor phenotypes are compatible (e.g., patient E+, donor E−), c) Mismatch—compared patient and donor phenotypes determined to be incompatible (e.g., patient E−, donor E+), d) Unknown—information is not available for a result to be determined (e.g., patient E− donor no information available for E antigen).

Demographics category match rule evaluation has one of the following three outcomes: a) True (e.g., patient race is black, donor race is black) b) False (e.g., patient race is white, donor race is black) c) Unknown—information is not available for a result to be determined (e.g., patient race is black; donor race is unknown).

Every match rule result is evaluated through the combination of two (2) Match Score parameters; 1) Match Score Code and 2) Priority Factor. Match Score Code represents the degree of matching. Priority Factor weighs the relative importance (impact) of a specific match rule.

In context, it is especially important to differentiate between ABO, D and K from all other antigen match rules when evaluating for exact match (MS0) and sufficient match (MS1).

The invention ranks antigens depending on their immunogenicity (ability to activate antibody production in the recipient's body) and clinical significance (ability to cause transfusion reactions and hemolytic disease of the fetus and newborn).

The Mismatch (MSM) Priority Factor is assigned according to antigen immunogenicity and antibody clinical importance.

Immune response with the introduction of an alloantigen does not always occur. Some antigens are very immunogenic while other antigens rarely cause an immune response. For example, most D−negative recipients produce anti−D antibodies after the administration of a D−positive RBC. Conversely, antigen s infrequently causes an immune response.

Priority Factor Examples

ABO System

| | | |
|---|---|---|
| Exact Match | 1^{st} choice | Priority Factor = 10,000 |
| Sufficient Match | 2^{nd} Choice | Priority Factor = 11,000 |
| Sufficient Match | 3^{rd} Choice | Priority Factor = 12,000 |
| Sufficient Match | 4th Choice | Priority Factor = 13,000 |
| Mismatch | Unacceptable | Priority Factor = 15,000 |

Rh, Kell, Duffy, Kidd, MNS Systems

| | | MS0 | MS1 | MSM |
|---|---|---|---|---|
| | | Priority | Priority | Priority |
| System | Antigen | Factor | Factor | Factor |
| Rh | D | 100 | 110 | 260 |
| Kell | K | 100 | 110 | 280 |
| Kell | k | 0 | 1 | 2 |
| Rh | C | 0 | 1 | 200 |
| Rh | c | 0 | 1 | 240 |
| Rh | E | 0 | 1 | 240 |
| Rh | e | 0 | 1 | 50 |
| Duffy | Fy^{a} | 0 | 1 | 220 |
| Duffy | Fy^{b} | 0 | 1 | 50 |
| Kidd | Jk^{a} | 0 | 1 | 240 |
| Kidd | Jk^{b} | 0 | 1 | 180 |
| MNS | S | 0 | 1 | 50 |
| MNS | s | 0 | 1 | 10 |
| MNS | M | 0 | 1 | 160 |
| MNS | N | 0 | 1 | 4 |
| Any Antibody | e.g., M | 0 | Not Applicable | 15000 |
| Race | Not Applicable | 0 | Not Applicable | 15000 |

Section 6: Patient Donor Matching in Match Score Definitions

Patient to donor matching process produces one of the following three outcomes: 1) Match, 2) Mismatch, 3) Unknown

1) Match: a) Exact Match−MS0, b) Sufficient Match−MS1.

Exact Match−MS0: Patient and donor phenotypes are identical and demographic rules are true. (e.g., patient phenotype: A, D+, K−, race is white, donor phenotype: A, D+, K−, race is white). Sufficient Match−MS1: Patient and donor phenotypes are compatible and demographics rules are true (e.g., patient phenotype: A, D+, K−, race is white, donor phenotype: A, D−, K, race is white).

2) Mismatch: Mismatch (MSM)

MSM: If at least one match rule returns MSM then patient to donor match result is considered MSM (e.g., patient phenotype: A, D+, K−, race is white, donor phenotype: A, D−, K+, race is white).

3) Unknown: Not enough information is available to evaluate. (Antigen K is missing in the example. e.g., patient phenotype: A, D+, K−, race is white, donor: A, D−, _, race is white).

Matching algorithm applies antigen matching rules as well as relevant demographic rules individually.

Patient Donor match result is represented in Match Score definitions:

Match Score Code

Match Score Code represents the degree of patient donor matching. The following logic is applied for determination of match score code.

Exact Match: If all applied match rules evaluated as MS0 (exact match) then Patient Donor Match Score Code=MS0 (exact match), B) Sufficient Match: If applied match rules evaluated as MS0 and MS1 (sufficient match) in any combination and no MSM (mismatch) results are present, then patient donor Match Score Code=MS1 (sufficient match), C) Mismatch: If any applied match rule is evaluated as MSM (mismatch) then patient donor Match Score Code=MSM (mismatch).

Priority Factor

Priority Factor quantitatively measures the match result. The patient donor Priority Factor is calculated as the sum of all applied match rules. The lowest Priority Factor is given the highest priority in donor selection.

However, Priority Factor works only as an additional Match Score parameter. Priority Factor can be used to identify a better donor match within the same matching outcome (the same Match Score Code).

Choice Factor

Choice Factor defines the deviation (number of rules) from an exact match for a patient donor match outcome (MS0, MS1). It is also the number of MSM (mismatch) rules in the case of a mismatch (MSM) situation.

Choice Depth

Choice Depth is the number of antigen rules applied in the matching algorithm.

Section 7: Patient Donor Matching—Better Match Determination

Match Score (MS) method uses deterministic rules to provide a mechanism to compare multiple donors to a patient and determine the best match. This method can also be used to determine “the best” incompatible donor. (e.g., A set of donors does not contain any compatible donors for a patient.)

There are four deterministic rules. Examples are shown in the ‘Illustrated Information’ chart below. 1) If match results are Exact Match (MS0) then donors are equal. (e.g., Donor A (#1) and Donor B (#2) are both best matches for this patient.) 2) If match results are Exact Match (MS0) and Sufficient Match (MS1) then MS0 donors are a better match. (e.g., donors A (#1), B (#2) are better than donor C (#3), D (#4) for this patient.) 3) If match results are Sufficient Match (MS1) then the lower the Priority Factor the better the match. (e.g., donor C (#3) is a better match than donor D (#4) for this patient. 4) If match results are Mismatch (MSM) then the lower the Priority Factor the lower the risk. (e.g., donor F (#5) is a lower risk than donor E (#6).)

Illustration Information (Patient to Donor Match Results—One Patient, Six Donors)

| | Patient | |
|---|---|---|
| # | Donor Pair | Match Result in Match Score Definitions |
| 1 | Patient - | Match Score = MS0, Priority Factor = 10200 |
| | Donor A | Choice Factor = 0, Choice Depth = 7 |
| 2 | Patient - | Match Score = MS0, Priority Factor = 10200 |
| | Donor B | Choice Factor = 0, Choice Depth = 7 |
| 3 | Patient - | Match Score = MS1, Priority Factor = 10201 |
| | Donor C | Choice Factor = 1, Choice Depth = 7 |
| | | Antigen C sufficient match, patient phenotype: C+, |
| | | donor C phenotype: C− |
| 4 | Patient - | Match Score = MS1, Priority Factor = 10202 |
| | Donor D | Choice Factor = 2, Choice Depth = 7 |
| | | Antigen C sufficient match, patient phenotype: C+, |
| | | donor D phenotype: C− |
| | | Antigen E sufficient match, patient phenotype: E+, |
| | | donor D phenotype: E− |
| 5 | Patient - | Match Score = MSM, Priority Factor = 10460 |
| | Donor E | Choice Factor = −1, Choice Depth = 7 |
| | | Antigen D mismatch, patient phenotype: D−, |
| | | donor E phenotype: D+ |
| 6 | Patient - | Match Score = MSM, Priority Factor = 10250 |
| | Donor F | Choice Factor = −1, Choice Depth = 7 |
| | | Antigen e mismatch, patient phenotype: e−, |
| | | donor F phenotype: e+ |

Section 8: Patient Donor Matching Example

Patient Profile

| Field | Value | | | | | | | | | | | | | | | |
|---|---|---|---|---|---|---|---|---|---|---|---|---|---|---|---|---|
| Name | Demo Patient | | | | | | | | | | | | | | | |
| Facility Patient ID | PDS2V1TRG2 | | | | | | | | | | | | | | | |
| Source Patient ID | 1 | | | | | | | | | | | | | | | |
| Gender | Male | | | | | | | | | | | | | | | |
| Age | 31 | | | | | | | | | | | | | | | |
| Race | White | | | | | | | | | | | | | | | |
| Transfusion Related | Blood (Hematological) Disorders (BD) | | | | | | | | | | | | | | | |
| Conditions | | | | | | | | | | | | | | | | |
| Assigned TRG | Transfusion Risk Group 2 (TRG 2) | | | | | | | | | | | | | | | |
| TRG Criteria | Females younger than 55, blood (Hematological) disorders, chronic transfusions due to | | | | | | | | | | | | | | | |
| | other diseases. | | | | | | | | | | | | | | | |
| TRG Choice Depth | 7: ABO, D, K, C, c, E, e (7 Antigen Rules) | | | | | | | | | | | | | | | |
| Recommendation | | | | | | | | | | | | | | | | |
| Message | Sufficient phenotype information available for donor selection | | | | | | | | | | | | | | | |
| Blood Type | A+ | | | | | | | | | | | | | | | |
| Phenotype Choice | 12 (Antigens information available) | | | | | | | | | | | | | | | |
| Depth | | | | | | | | | | | | | | | | |
| Phenotype | ABO | D | C | c | E | e | K | k | Jk^{a} | Jk^{b} | Fy^{a} | Fy^{b} | M | N | S | s |
| | A | + | + | − | − | + | − | + | + | − | + | − | N/A | N/A | N/A | N/A |

Patient Donor Compatibility and Patient Donor Match Explained

The Patient Donor Final Compatibility determination is based on the patient's assigned TRG (TRG 2). TRG 2 requires phenotype information of seven (7) antigen rules (Choice Depth=7).

The Patient Donor Final Match is based on the patient's available phenotype information. Information on 12 antigen is available (Choice Depth=12).

The Donor phenotype information is available on 16 antigens (Choice Depth=16).

| Phenotype | ABO | D | C | c | E | e | K | k | Jk^{a} | Jk^{b} | Fy^{a} | Fy^{b} | M | N | S | s |
|---|---|---|---|---|---|---|---|---|---|---|---|---|---|---|---|---|
| Patient | A | + | + | − | − | + | − | + | + | + | + | − | N/A | N/A | N/A | N/A |
| Donor | A | + | + | − | − | + | − | + | − | + | + | − | − | + | + | − |

Patient Donor Matching Example Explained

Lines 3-9,14 Phenotype: ABO, D, K, C, c, E, e (7 Antigen Rules)

Line 1: Patient Donor Final Compatibility=MS0

Line 3-9,14 Exact match, patient and donor antigen sets are identical based on the required information as specified by recipient's Transfusion Risk Group

Line 2: Patient Donor Final Match=MS1

Line 3-9,14: Sufficient match based on all available information

Line 10-13: Antigens Jk^{a}, Jk^{b}, Fy^{a}, Fy^{b} are not included in TRG2 definition

TABLE
Patient Donor Matching Example

| | | | | | Match Score (MS) Method Factor | | | |
|---|---|---|---|---|---|---|---|---|
| Line | | | | Match | Match | Priority | Choice | Choice |
| # | Patient | Donor | Match Rule | Category | Score Code | Factor | Factor | Depth |
| 1 | Patient | Donor | Final Compatibility | Compatibility | MS0 | 10200 | 0 | 7 |
| 2 | Patient | Donor | Final Match | Compatibility | MS1 | 10201 | 2 | 12 |
| 3 | Patient | Donor | ABO | Phenotype | MS0 | 10000 | 0 | 1 |
| | | | (ABO System) | | | | | |
| 4 | Patient | Donor | Rh(D) | Phenotype | MS0 | 100 | 0 | 1 |
| | | | (D antigen) | | | | | |
| 5 | Patient | Donor | Kell(K) | Phenotype | MS0 | 100 | 0 | 1 |
| | | | (K antigen) | | | | | |
| 6 | Patient | Donor | Rh(C) | Phenotype | MS0 | 0 | 0 | 1 |
| | | | (C antigen) | | | | | |
| 7 | Patient | Donor | Rh(c) | Phenotype | MS0 | 0 | 0 | 1 |
| | | | (c antigen) | | | | | |
| 8 | Patient | Donor | Rh(E) | Phenotype | MS0 | 0 | 0 | 1 |
| | | | (E antigen) | | | | | |
| 9 | Patient | Donor | Rh(e) | Phenotype | MS0 | 0 | 0 | 1 |
| | | | (e antigen) | | | | | |
| 10 | Patient | Donor | Kidd (Jka) | Phenotype | MS1 | 1 | 1 | 1 |
| | | | (Jk^{a} antigen) | | | | | |
| 11 | Patient | Donor | Kidd (Jkb) | Phenotype | MS0 | 0 | 0 | 1 |
| | | | (Jk^{b} antigen) | | | | | |
| 12 | Patient | Donor | Duffy (Fya) | Phenotype | MS0 | 0 | 0 | 1 |
| | | | (Fy^{a} antigen) | | | | | |
| 13 | Patient | Donor | Duffy (Fyb) | Phenotype | MS0 | 0 | 0 | 1 |
| | | | (Fy^{b} antigen) | | | | | |
| 14 | Patient | Donor | Race | Demographics | MS0 | 0 | 0 | Not Applicable |

Section 9: RBC Blood Unit Factors in Match Score Definitions

Red blood cell unit information contains expiration date, current storage method, and donor.

Unit inherits donor's phenotype and phenotype usage type (PUT).

Priority Factor is a measurement of blood unit specific factors.

Unit Specific Factors are long-term storage method (cryopreservation) and Extraordinary PUT.

Measurement of Blood Unit Specific Factors

| Unit Specific Factor | Priority Factor |
|---|---|
| Cryopreserved RBC unit (long-term storage) | 300 |
| Donor Extraordinary phenotype usage type | 5000 |

When evaluating a patient RBC unit match, Unit Specific Factors are considered in addition to a donor patient match result.

Section 10: Patient RBC Unit Matching and Ranking

Patient Unit Score (match result) is represented by Match Score (MS) method parameters: Match Score Code, Priority Factor, Choice Factor, and Choice Depth.

Patient Unit Match Score Code, Choice Factor, and Choice Depth are inherited from the Patient Donor Match result.

The Patient Unit Priority Factor is calculated by adding Unit Specific Factors to the Patient Donor Priority Factor.

The invention compares multiple donor blood units for a patient and determines the best match using Match Score Code and Patient Unit Priority Factor.

Patient Unit Ranking

The invention assigns Unit Selection Rank using Match Score Code and patient unit Priority Factor.

Patient Unit Selection Scores and Ranking Examples

### EXAMPLE 1

Patient 1— Transfusion Risk Group 4 (TRG 4)

| | Unit | | Phenotype | | Unit | Donor | Donor | Unit | Unit |
|---|---|---|---|---|---|---|---|---|---|
| | Expiration | | Usage Type | Storage | Additional | Match | Priority | Priority | Selection |
| Unit | Date | Product | (PUT) | Method | Factor | Score | Factor | Factor | Rank |
| 1 | Apr. 15, 2021 | RBC | Common | Refrg | 0 | MS0 | 10200 | 10200 | 1 |
| 2 | May 10, 2021 | RBC | Common | Refrg | 0 | MS0 | 10200 | 10200 | 2 |
| 3 | May 12, 2030 | RBC | Unique | Cryo | 300 | MS1 | 10201 | 10501 | 3 |
| 4 | Feb. 19, 2021 | RBC | Common | Refrg | 0 | MSM | 11160 | 11160 | Not |
| | | | | | | | | | Applicable |

### EXAMPLE 2

Patient 2— Transfusion Risk Group 2 (TRG 2)

| | Unit | | Phenotype | | Unit | Donor | Donor | Unit | Unit |
|---|---|---|---|---|---|---|---|---|---|
| | Expiration | | Usage Type | Storage | Additional | Match | Priority | Priority | Selection |
| Unit | Date | Product | (PUT) | Method | Factor | Score | Factor | Factor | Rank |
| 1 | Apr. 15, 2021 | RBC | Common | Refrg | 0 | MS0 | 10200 | 10200 | 1 |
| 2 | May 10, 2021 | RBC | Common | Refrg | 0 | MS0 | 10200 | 10200 | 2 |

Units that are compatible with the patient (with match scores of MS0 or MS1) are ranked by ‘Unit Priority Factor’.

Patient Unit Recommendation

Invention optimizes the decision-making task across multiple patients by assigning a recommended unit priority rank (Unit Recommendation) to each patient unit pair.

TRGs are prioritized in the order; TRG 6, TRG5, TRG4, TRG3, TRG2, TRG1 with the highest priority being given to TRG 6 and lowest priority to TRG 1.

In the example below Patient 1 (TRG 4) has a higher priority than Patient 2 (TRG 2).

Patient Unit Recommendation is the best patient unit match (highest ranking unit for a patient). Units that are ‘In bank inventory’ but claimed by another patient are excluded.

Patients 1 and 2— Recommendation Example

| | | | Unit | Unit | |
|---|---|---|---|---|---|
| Line | | | Selection | Recommen- | |
| # | Patient | Unit | Rank | dation | Comment |
| 1 | Patient 1 | 1 | 1 | 1 | Recommended |
| | (TRG 4) | | | | RBC unit |
| 2 | | 2 | 2 | 2 | |
| 3 | | 3 | 3 | 3 | |
| 4 | Patient 2 | 1 | 1 | Not | RBC unit taken by |
| | (TRG 2) | | | Available | Patient 1 |
| 5 | | 2 | 2 | 1 | Recommended |
| | | | | | RBC unit |

Section 11: The Phenotype Usage Type (PUT) Classification

Cryopreservation, storage and thawing of blood components are expensive procedures. Therefore, red blood cells from a long-term storage bank are transfused only in special cases where there is an absence of compatible refrigerated red blood cells and timing does not permit inviting suitable donors. Cryopreservation blood banks also provide a repository of red blood cells whose phenotype does not contain antigens or combinations of antigens often found in the population.

When managing a long-term storage reserve, it is necessary to maintain an equilibrium between a high probability of finding red blood cells with the requested phenotype, and the excessive accumulation of red blood cells that go unclaimed and ultimately expire.

The Phenotype Usage Type (PUT) classification scheme is introduced to assist in solving this challenge. PUT describes how RBC blood units should be managed. PUT is associated with a developed algorithm for the optimization of blood banking operations.

The PUT classification introduces the Unique and Extraordinary categories and identifies phenotypes that belong to them.

The complete PUT classification scheme includes Unique, Extraordinary, Required, Universal, and Common categories.

Each PUT category implies the analysis of certain antigens in the donor phenotype.

The Unique Phenotype covers antigens of ABO, Rh (D, C, c, E, e), Kell (K, k), Kidd (Jk^{a}, Jk^{b}), Duffy (Fy^{a}, Fy^{b}), MNS (M, N, S, s) systems. Red blood cells of this level of selection are primarily transfused to recipients of TRG 3, 5, 6.

The Required Phenotype contains ABO, Rh (D, C, c, E, e), Kell (K) antigens. This level of compatibility is necessary for patients TRG2 and in some cases TRG4.

The Universal Phenotype includes ABO, Rh (D, C, c, E, e), Kell (K) antigens. Red blood cells of this level of selection are transfused to recipients of TRG 0, 1, 2, 4.

The Extraordinary Phenotype does not have common antigens or antigenic combinations in its composition.

Common Phenotype does not belong to any of the above categories.

Unique Category (PUT)

Phenotypes classified as Unique are most compatible with recipient phenotypes of the clinically relevant antigens of ABO, Rh, Kell, Kidd, Duffy and MNS blood group systems.

Additionally, RBCs classified as Unique express only one antigen in the pairs: C/c, E/e, Fy^{a}/Fy^{b}, Jk^{a}/Jk^{b}, S/s, M/N. Because such combinations provide compatibility with recipients whose genotype contain genes encoded with antigens in either homozygous or heterozygous states.

In addition, red blood cells with phenotypes classified as Unique possess neither antigens A and B of the ABO system, nor K of the Kell system.

The Unique category also includes a subset of phenotypes where the Fy^{a} or Fy^{b} antigens of the Duffy system are absent.

There are 80 Unique phenotypes which are present for only a small number of donors. However, Unique phenotypes are compatible with 99.8% of recipients for Choice Depth=16 (16 antigens: AB, D, C, c, E, e, K, k, Jk^{a}, Jk^{b}, Fyₐ, Fy^{b}, M, N, S, s).

The recommended standard of care of the Unique PUT category is to cryopreserve RBC units.

Note: The C^{w} antigen has been excluded from the Unique phenotype definition, as scientific schools differ in their approach on the C^{w} antigen.

The C^{w} antibody is clinically significant. Should the C^{w} antibody be present in a recipient, the invention's matching algorithm will consider only donor phenotypes that are C^{w}— (C^{w} absent).

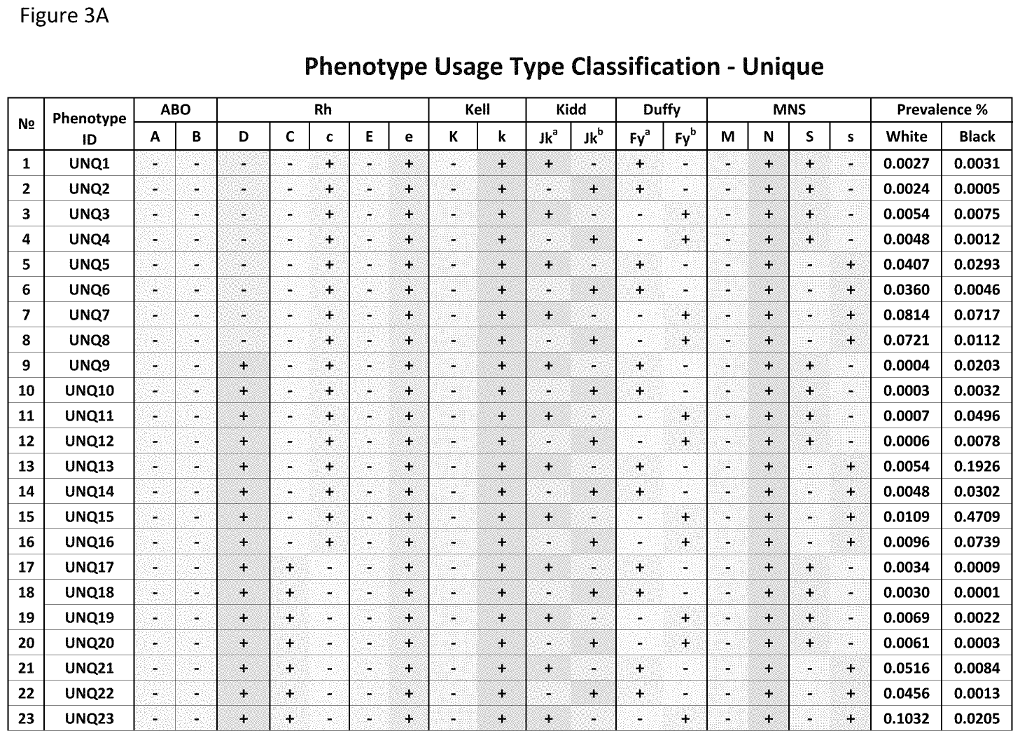

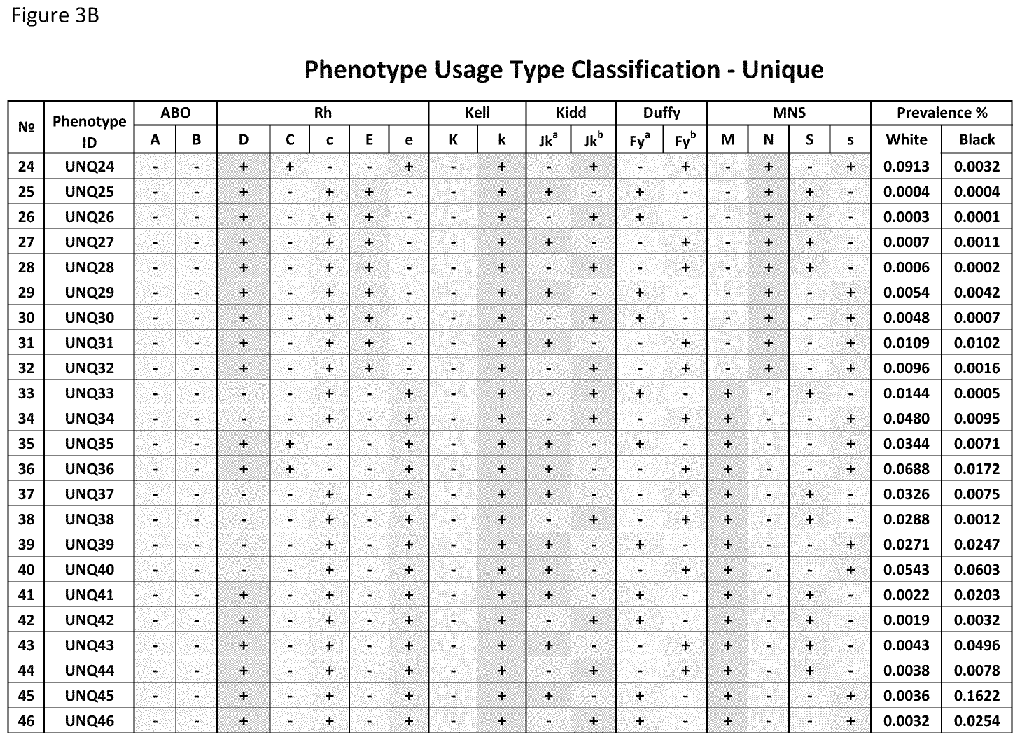

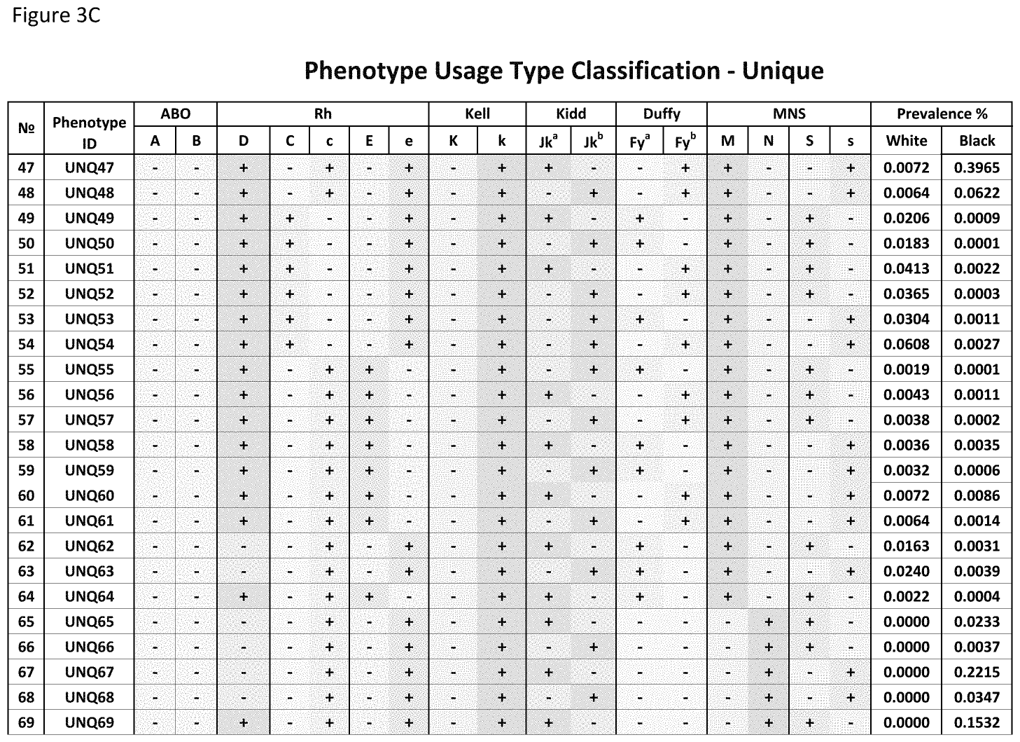

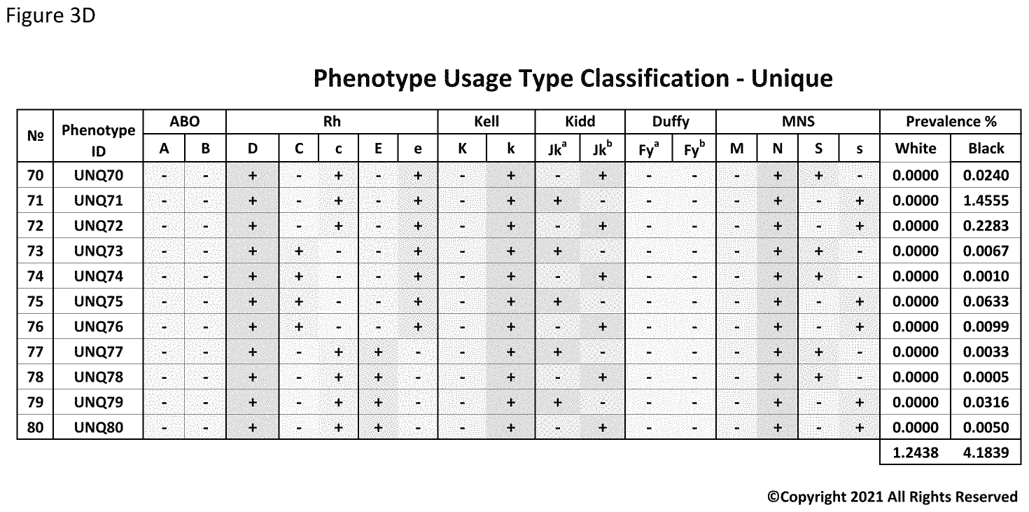

Refer to FIGS. 3A, 3B, 3C, and 3D for Unique Phenotype Usage Type Classification. These Figures show the combinations of antigens that determine the Unique PUT Category. Prevalence information as per Reid M. E et al.¹⁷ and Shaz B. H et al.¹⁸

Required Category (PUT)

Phenotypes classified as Required are in strong demand for both planned and emergency transfusions.

The Required phenotype category considers the compatibility of the donor and recipient for ABO, Rh (D, C, c, E, e), and Kell (K) blood group systems.

The recommended standard of care of the Required PUT category is to maintain a minimal supply level of refrigerated RBC units.

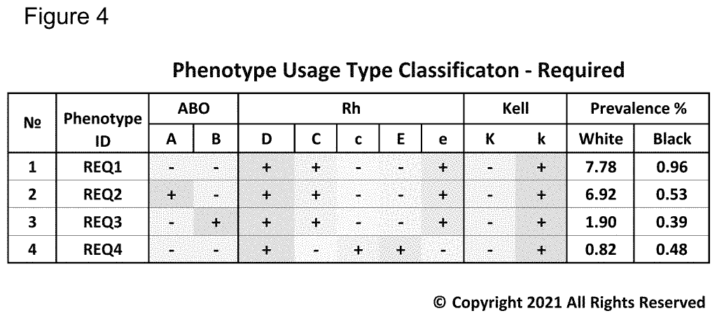

Refer to FIG. 4 for Required Phenotype Usage Type Classification. Figure shows the combinations of antigens that determine the Required PUT Category.

Universal Category (PUT)

Phenotypes classified as Universal are used for immediate transfusions to recipients whose blood type is not determined as well as for scheduled transfusions to recipients compatible with ABO and Rh group systems.

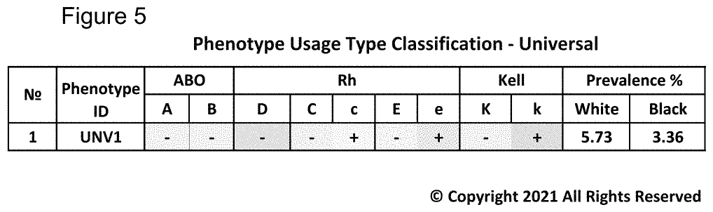

Refer to FIG. 5 for Universal Phenotype Usage Type Classification. Figure shows the combinations of antigens that determine the Universal PUT Category.

Extraordinary Category (PUT)

A phenotype is classified as Extraordinary when it lacks an antigen or antigen combination typically found in greater than 99% of the population and it has an optimal combination of ABO and Rh antigens.

The recommended standard of care is to have 1-2 RBC units of each Extraordinary phenotype cryopreserved.

RBCs of these phenotypes are rarely used because they are not compatible with most recipient phenotypes. However, they may be required for patients who are alloimmunized to most common antigens.

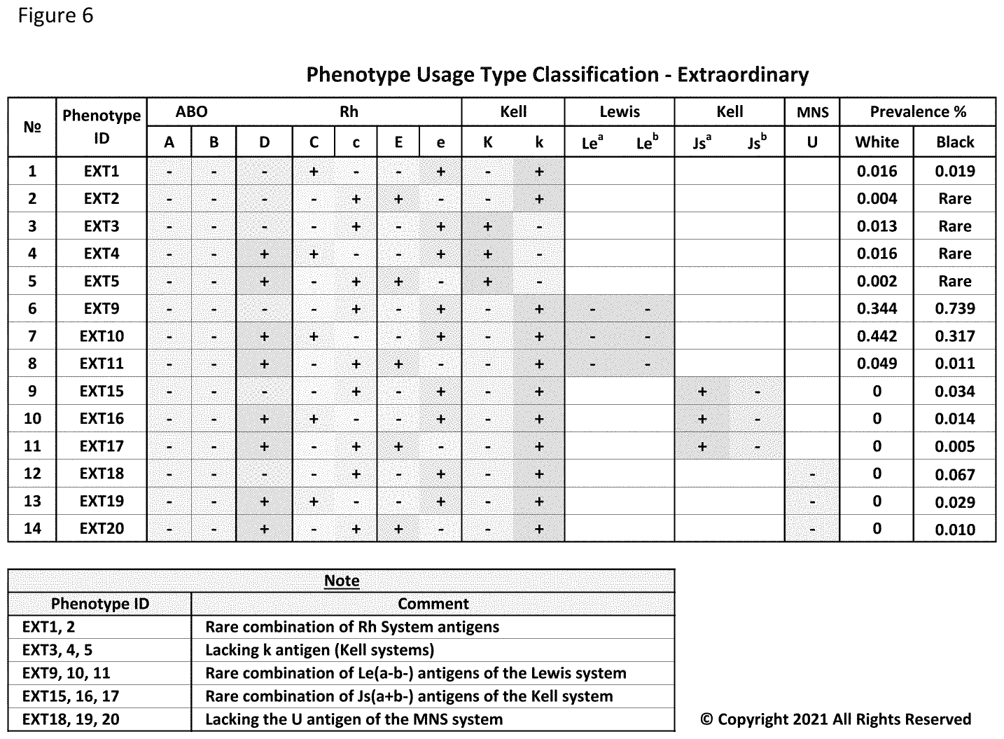

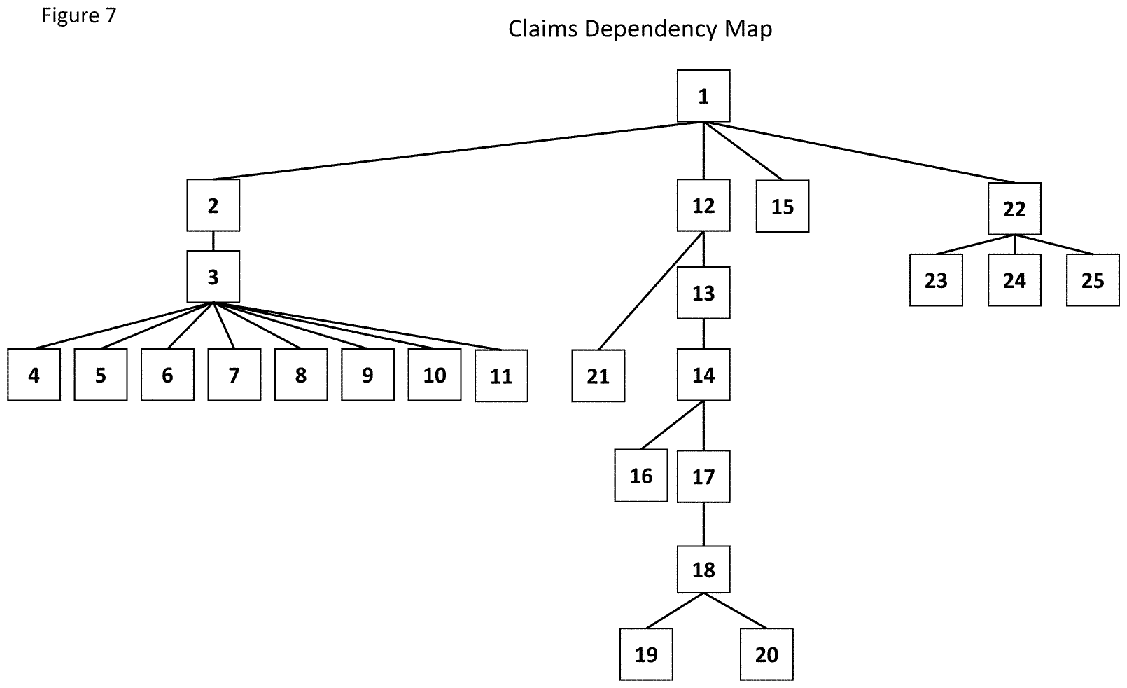

Refer to FIG. 6 for Extraordinary Phenotype Usage Type Classification. Figure shows the combinations of antigens that determine the Extraordinary PUT Category.

Section 12: Blood Bank Cryopreservation Optimization

The Phenotype Usage Type (PUT) Concept is introduced to assist in performing the blood bank RBC cryopreservation optimization task (see Detailed Description; Section 11: The Phenotype Usage Type (PUT) Classification for detail).

In accordance with the PUT classification there are two (2) Phenotype Usage Type units that are recommended to be cryopreserved, they are Unique and Extraordinary.

The blood cryopreservation optimization algorithm identifies refrigerated Unique RBC units, flags them, and recommends changing their storage method to cryopreservation.

The blood cryopreservation optimization algorithm identifies refrigerated Extraordinary RBC units, checks if this specific Extraordinary RBC phenotype is already in the blood bank inventory in a cryopreserved form and if it is not recommends changing the storage method to cryopreservation.

Blood Bank Cryopreservation Optimization Example

| | | Phenotype | | |
|---|---|---|---|---|
| | Storage | Usage Type | Phenotype | |
| Unit | Method | (PUT) | ID | Recommendation |
| 1 | Refrigerated | Unique | UNQ12 | Cryopreserve RBC unit |
| 2 | Refrigerated | Unique | UNQ25 | Cryopreserve RBC unit |
| 3 | Refrigerated | Extra- | EXT5 | Cryopreserve RBC unit |
| | | ordinary | | Blood bank does not |
| | | | | have cryopreserved |
| | | | | EXT5 Extraordinary |
| | | | | PUT. |
| 4 | Refrigerated | Extra- | EXT7 | No action |
| | | ordinary | | Blood bank already has |
| | | | | cryopreserved EXT7 |
| | | | | Extraordinary PUT. |

### Glossary

It will be appreciated that for the sake of clarity and simplicity that certain key words and phrases be defined which will be used throughout.

Alloimmunization—Process of antibody formation in response to antigenic stimulation.

Alloantibody—Antibody aimed at the antigens of blood cells that are missing from the recipient.

Antigens—Structural components of cell membranes, genetically determined. They have immunogenicity and form an antigen-antibody complex. The International Society of Blood Transfusion (ISBT) terminology is used for the antigens.

Autoantibody—Antibody aimed at its own blood cell structure.

Blood Group (The phenotype of RBCs)—A collection of red blood cell antigens of an individual indicating their presence or absence in the testing result.

Blood Group System (BGS)—System used to group human blood into different types, based on the presence or absence of certain markers (antigens) on the surface of red blood cells. The International Society of Blood Transfusion (ISBT) terminology is used for the blood group systems.

Blood Product Unit—Whole blood or one or more blood components, such as but not limited to red blood cells (RBC), white blood cells (WBC), blood plasma, and platelets. Blood units are often packaged for storage and distribution in standard size containers, packaging and unit size standards vary.

Blood Transfusion—The injection of a blood product that originated from a donor into a patient.

Compatibility—In the context of blood transfusion, compatibility denotes a match between the donor's blood and the recipient's blood.

Chimera (chimerism)—In medicine, a person composed of two genetically distinct types of cells.

Clinically significant alloantibodies—Antibodies that can cause transfusion hemolytic reactions and/or hemolytic disease of the fetus and newborn.

Donor—Provider and source of a blood product unit.

IHF-Immunohematology Framework—is the working name of the invention.

Extended Phenotype Matching (also defined as Red Cell antigen typing other than Rh (D, C, c, E, e) and Kell (K) antigens)—Determination the presence (or absence) of specific red cell antigens which includes, but not limited to: Fy^{a}, Fy^{b}, Jk^{a}, Jk^{b}, S, s.

Limited Phenotype Matching—Determination the presence (or absence) of specific red cell antigens which includes ABO, Rh (D, C, c, E, e), K

Match Score (MS)— A (introduced) method to provide the capability to quantitatively define the match results of patient and donor compatibility, and to calculate patient unit selection score, and to measure and compare unit selection decisions to ensure the optimal level of compatibility between blood transfusion recipients and donors while optimizing blood products distribution.

Panagglutination—the ability of serum to agglutinate all red blood cell samples, regardless of their phenotype.

Patient—Recipient and receiver of a blood product unit.

Phenotype Usage Type (PUT) —A (introduced) classification scheme that describes how RBC blood units should be managed. PUT is associated with a developed algorithm for the optimization blood banking operations.

RBC Phenotyping—Refers to determining the type of antigens present on the RBC.

Transfusion Risk Group (TRG)—A (introduced) classification scheme that combines relevant patient demographics and transfusion conditions. TRG concept assigns patients into groupings indicating the required selection level by phenotype.

Abbreviations Used

BGS Blood Group System

Cryo Cryopreserved

CML Chronic Myeloid Leukemia

EMR Electronic Medical Records

HDFN Hemolytic Disease of Fetus and Newborn

HTR Hemolytic Transfusion Reactions

IHF Immunohematology Framework

MS Match Score

MDS Myelodysplastic Syndrome

PUT Phenotype Usage Type

RBC Red Blood Cell

Refrg Refrigerated

SCD Sickle Cell Decease

THA Thalassemia

TRG Transfusion Risk Group

### CITATIONS

1. García-Roa M., Vicente-Ayuso M., Bobes A., Pedraza A., González-Fernandez A., Martín M., Sáez I., Seghatchian J., Gutiérrez L. Red blood cell storage time and transfusion: current practice, concerns and future perspectives. Blood Transf. 2017 May; 15(3): 222-231
2. Yoshida T., Prudent M., D'alessandro A. Red blood cell storage lesion causes and potential clinical consequences. Blood Transfus. 2019 Jan;17(1):27-52.
3. Annual SHOT Report. Available online at: https://www.shotuk.org/shot-reports/4.
4. Stiegler G., Sperr W., Lorber C. et al. Red cell antibodies in frequently transfused patients with myelodysplastic syndrome. Ann Hematol. 2001 June; 80(6):330-3.
5. Zalpuri S., Zwaginga J. J., le Cessie S. et al. Red-blood-cell alloimmunization and number of red-blood-cell transfusions. Vox Sang. 2012 February;102(4):144-9.
6. Schonewille H., Haak H. L., van Zijl A. M. Alloimmunization after blood transfusion in patients with hematologic and oncologic diseases. Transfusion.1999 Jul; 39(7):763-71.
7. Ortiz S., Orero M. T., Javier K. et al. Impact of azacitidine on red blood cell alloimmunization in myelodysplastic syndrome. Blood Tranfus. 2017 September; 15(5):472-477.
8. Novaretti M. C., Sopelete C. R., Velloso E. R. et al. Immunohematological findings in myelodysplastic syndrome. Acta Haematol. 2001; 105(1):1-6.
9. Leisch M., Weiss L., Lindlbauer N. et al. Red blood cell alloimmunization in 184 patients with myeloid neoplasms treated with azacitidine— A retrospective single center experience. Leuk Res. 2017 August; 59:12-19.
10. Davis B. A., Allard S., Qureshi A. et al. Guidelines on red cell transfusion in sickle cell disease Part II: indications for transfusion. Br. J. Haematol. 2017; 176(2):192-209.
11. Sanz C., Nomdedeu M., Belkaid M. et al. Red blood cell alloimmunization in transfused patients with myelodysplastic syndrome or chronic myelomonocytic leukemia. Transfusion. 2013 April; 53(4):710-5.
12. Tormey A. Christopher., Hendrickson E Jeanne. Transfusion-Related Red Blood Cell Alloantibodies: Induction and Consequences. Blood. 2019 Apr. 25; 133(17):1821-1830.
13. Fasano R. M., Meyer E. K., Branscomb J. et al. Impact of Red Blood Cell Antigen Matching on Alloimmunization and Transfusion Complications in Patients with Sickle Cell Disease: A Systematic Review. Transfus Med Rev. 2019 January; 33(1):12-23.
14. Lasalle-Williams M., Nuss R., Le T. et al. Extended red blood cell antigen matching for transfusions in sickle cell disease: a review of a 14-year experience from a single center (CME). Transfusion.2011 Aug; 51(8):1732-9.
15. Gehrie E. A., Tormey C. A. The Influence of Clinical and Biological Factors on Transfusion-Associated Non-ABO Antigen Alloimmunization: Responders, Hyper-Responders, and Non-Responders. Transfus Med Hemother. 2014 November; 41(6):420-9.
16. Van Sambeeck J. H. J., de Wit P. D., Luken J. et al. A Conceptual Framework for Optimizing Blood Matching Strategies: Balancing Patient Complications Against Total Costs Incurred. Front. Med. (Lausanne). 2018 July; 5:199.
17. Reid M. E., Lomas-Francis C., Olsson M. L. The Blood Group Antigen Facts Book. 3rd ed. New York: Academic Press; 2012.
18. Shaz B. H., Hillyer C. D., Roshal M.et al. Transfusion Medicine and Hemostasis: Clinical and Laboratory Aspects, 2nd ed.- Philadelphia, Pa.: Elsevier; 2013.

What is claimed is:

1. A method for blood transfusion information analysis for the purpose of recommending specific blood product units/donors for transfusion candidates including those of complex cases, rare blood types and emergent situations to achieve: a better patient—donor match, prevention of alloimmunization and more effective prevention of transfusion hemolytic reactions, as well as improved optimization of the long-term storage of red blood cell units; a classification scheme to be used to arrange patients into groups indicating the required antigen selection level by phenotype for optimal donor match, explaining which demographic, medical, and laboratory information should be considered, and assessing the sufficiency of a patient's available blood typing information; determination and measurement of patient donor compatibility, measurement and comparison of unit selection decisions; a classification scheme to be used to arrange donors by phenotype enabling optimization of unit selection for cryopreservation, and for maintaining an equilibrium between a high probability of finding requested red blood cell phenotypes and the excessive accumulation of red blood cell units that go unclaimed and ultimately expire.

2. The method of claim 1 to classify patients into groupings to optimize donor selection to prevent the formation of unexpected antibodies so that the patient does not transition to an alloimmunized state, and to minimize the possibility of hemolytic transfusion reactions; this method combines relevant demographics and medical conditions taking into consideration: a) clinical criteria: blood diseases, long-term transfusion therapies, and drugs that cause nonspecific red blood cell agglutination, b) laboratory conditions: the presence of specific and multispecific antibodies, and c) demographics: woman's age in the absence of relevant clinical and laboratory conditions.

3. The method of claim 2 to classify a patient into one of seven (7) transfusion risk groups where each group has its own required blood typing criteria: a) four transfusion risk groups are defined according to the required antigen matching rules, b) two groups consider unexpected specific antibodies in the matching process.

4. The method of claim 3 to classify a patient who is a male or female over 55 years of age and possess no negative transfusion conditions into transfusion risk group one; donor selection is required to be based on phenotype matching for three transfusion dangerous antigens: ABO, D, K.

5. The method of claim 3 to classify a patient who has a high probability of future transfusions or pregnancies into transfusion risk group two; donor selection is required to be based on limited phenotype matching for seven transfusion dangerous antigens: ABO, D, K, C, c, E, e.

6. The method of claim 3 to classify a patient who has a lifelong blood transfusion dependency into transfusion risk group three; donor selection is required to be based on extended phenotype matching for thirteen transfusion dangerous antigens: ABO, D, K, C, Fy^{a}, Fy^{b}, Jk^{a}, Jk^{b}, S, s.

7. The method of claim 3 to classify a patient who has unexpected specific antibodies into transfusion risk group four; donor selection is required to be based on phenotype matching for seven transfusion dangerous antigens: ABO, D, K, C, c, E, e and antigens which correspond to one or more specific antibodies the patient possesses including: anti−D, −C, −c, −E, −e, −K, −k, −Fy^{a}, −Fy^{b}, −Jk^{a}, −Jk^{b}, −S, −s, −M, −N, −Lu^{a}, −Lu^{b}, −Le^{a}, −Le^{b}, −Kp^{a}, −Kp^{b}, −Js^{a}, −Js^{b}, −C^{w}.

8. The method of claim 3 to classify a patient who has unexpected specific antibodies and a clinical blood system disease(s) into transfusion risk group five; donor selection is required to be based on extended phenotype matching for thirteen transfusion dangerous antigens: ABO, D, K, C, Fy^{a}, Fy^{b}, Jk^{a}, Jk^{b}, S, s and antigens which correspond to one or more specific antibodies the patient possesses including: anti−D, −C, −c, −E, −e, −K, −k, −Fy^{a}, −Fy^{b}, −Jk^{a}, −Jk^{b}, −S, −s, −M, −N, −Lu^{a}, −Lu^{b}, −Le^{a}, −Le^{b}, −Kp^{a}, −Kp^{b}, −Js^{a}, −Js^{b}, −C^{w}.

9. The method of claim 3 to classify a patient who has multispecific antibodies into transfusion risk group six; donor selection is required to be based on extended phenotype matching for 14 transfusion dangerous antigens: ABO, D, K, C, c, E, e, Fy^{a}, Fy^{b}, Jk^{a}, Jk^{b}, S, s, M.

10. The method of claim 3 to classify a patient who requires an immediate transfusion and phenotype information is unavailable into transfusion risk group zero; donor selection should be of phenotype: O, D−C−c+E−e+, K− (O, ccdee, K−).

11. The method of claim 3 to determine if patient's blood typing information is sufficient to perform donor matching. This is determined by evaluating patient's available phenotype information against the patient's transfusion risk group antigen requirements.

12. The method of claim 1 to evaluate the decision of a manually completed blood unit selection against the invention recommendation and to measure its quality by comparing the patient unit match scores and difference in priority factors.

13. The method of claim 12 to determine and measure patient unit compatibility and rank patient compatible units; patient compatible units are ordered by the value of the patient unit priority factor which is calculated by adding unit additional factors to patient donor priority factor; the highest-ranking available unit is recommended for the patient.

14. The method of claim 13 to determine and measure patient donor compatibility is based on the required demographic, phenotype, antibody information as specified by the patient's transfusion risk group.

15. The method of claim 1 to determine and measure patient donor match based on all available demographic, phenotype, antibody information.

16. The method of claim 14 to consider additional patient donor matching logic for immunohematological conditions such as chimerism, and the presence of relevant alloantibodies and autoantibodies.

17. The method of claim 14 provides the capability to quantitatively define the match results of patient donor compatibility, calculate patient unit selection score, measure, and compare unit selection decisions.

18. The method of claim 17 is expressed by the match score code which represents the degree of matching a) exact match which represents identical patient and donor phenotypes, b) sufficient match which represents compatible patient and donor phenotypes, c) mismatch which represents incompatible patient and donor phenotypes.

19. The method of claim 18 is clarified by the priority factor which is a numerical measurement of a) an executed fine grain match rule result, b) a patient donor compatibility, c) a patient donor match d) a patient unit selection score, e) blood unit specific factors, and f) patient demographic relevant characteristics.

20. The method of claim 18 is supplemented by the choice factor which defines the deviation from an exact match, as well as by choice depth which defines the number of antigen rules applied in matching.

21. The method of claim 13 to determine and measure unit specific factors is based on the storage method, expiration date, and phenotype usage type.

22. The method of claim 1 to classify donor phenotypes into five (5) phenotype usage type categories, each which describes how red blood cell units of that category should be stored and managed for optimal blood bank operations for the purpose of maintaining a blood bank long-term storage equilibrium between a high probability of finding red blood cells with the requested phenotype and the excessive accumulation of red blood cells that go unclaimed and ultimately expire.

23. The method of claim 22 to classify donor phenotypes in a unique category which are most compatible with recipient phenotypes of the 16 clinically relevant antigens of ABO, Rh, Kell, Kidd, Duffy and MNS blood group systems; these unique phenotypes contain only one antigen in the pairs: C/c, E/e, Fy^{a}/Fy^{b}, Jk^{a}/Jk^{b}, S/s, M/N; additionally, these phenotypes must not possess antigens A and B of the ABO system and K of the Kell system; also a subset of these unique phenotypes have the Fy^{a} or Fy^{b} antigens of the Duffy system absent.

24. The method of claim 22 to classify donor phenotypes in an extraordinary category which are rarely used because they are not compatible with most recipient phenotypes, however, they may be required for patients who are alloimmunized to most common antigens.

25. Method of claim 22 to determine the recommended storage method of red blood cell units using phenotype classification; long term storage for every unit of unique phenotype and one (1) unit of extraordinary phenotype.
